SEQUENCE LISTING (11 sequences)

SEQ ID NO: 1 - DNA, 582 bp, bacteroides
cccttcgtaatagttccgccacgaagcaagatttcattgttttcacctattttagcctctactcccggaagcaaacgtcccacggcaccgatgcggaaatcatctttccattcacaagacaccgtagccgtactttctgtcaatccatagccggctaccatgttaatacctaccgaatgaacaaattcagccacctttccaggaatagccgcaccggcagtcgggaaaaagtttccgttgtcaattcctatcgtctttttcaggaggctgtaaatcgtccgttcataaaacttatatttcatatgcaacatcacgggaggagtcttaccattcatcaggtaatcaatgttatgcgccttacctacacgaagggcatccaacatcagactctttttcagccccgttgtttcgttaatcttttccagtacgcccgcgtagactttttcccagaaacggggcacactgcacatggctgtagggcgaacctccttgatggttttaatatcctgcggacggaggttaatgcagagcaaacatccacggtctaaacaataatatgaccatgcccgctcaaaaatggta

SEQ ID NO: 2 - DNA, 355 bp, bacteroides
ggcacgtagagaatacgaaggtgagcgagagaactgttgccaaggaactcggcaaaatggccccgtacgttcgcaagaaggggcgctcgagagagccgcagtgaaaaggcccaagcgactgtttaactaaaacacagctctatgcaaagccgtaaggcgaagtatatggggtgacacctgcccggtgctggaaggttaaggggatctgttcgtcgcaagacaaagcagtggtccgaagccccagtgaacggcggccgtaactataacggtcctaaggtagcgaaattccttgtcgggtaagttccgacccgcacgaaaggcgtaatgatttgagcgctgtctcgacaatccagtg

SEQ ID NO: 3 - DNA, 550 bp, bacteroides
tgaaataagaaccagggaaaaccttgaggaaaaggagctatcatagctgacgcatatcaggaacactgataccgttcacgcccagtctgcgcttcctttgagcctgtagtctcaaaggcgctcgacggattcgcgtaaacggtgtcaatgtcccggatatgctgttggcatgatagctctttgtgtataggtgtgcagatggagagtgtgtttgaataaatcgaaaaagtggaggcggattatggaagagtgtatgacaaagaggggagccaggctgattgtgtatgtgcctaaggagctggatcatcattttgcaggggagatgacagagatggtggataaggagcttaagaagggctgtattaaacagcttgtctttgatttttctaagacaacgtttatggacagttctggcattgggatgctaatggggagaaagcggctgttaagctacagcggaggaaccgtaagtgctattcatgttaatgaccggattttgcggattatgcagccttcgggcatctggaaacacatggaaatcagccaggaa

SEQ ID NO: 4 - DNA, 416 bp, bacteroides
tagtccttttctcccacggtcatctgctctttcatactgtccataagaagctgcttacattcctgtggatccttaatatgcttttctgccacctgctcctttaaacgttcaataatagaagtggtagtattaattccaatgtcgcccataaccaagatttcttcgatttcctcatagaaatcctcgtcaatactggaataaccgctaaaaatagaatcaattcctgatacaatgttttctctggtttttgaaagaccctctaccaatcttttaaaaaaaccttttttctcttttccctctgccatagttcctcctatttatctaaatcatgctcaatgagttttacagacaccagggtagacacacctttttcctgcatggtaattccatagagcctgtctgctgctgtcatggtt

SEQ ID NO: 5 - DNA, 387 bp, bacteroides
gtgctgaccaaggaggtgagaagcagcactatcaaggcaattttaagaatcagcttgcgccgattgattatgtcagatggatgaagcatagtgactgtgtaacttaaatgggtgttcagaattcacacatttgaacacttgcttgggtaacttgaactttaaactcgatgcttaaaacgcctgaatggtgtaaaggtaaagaacggctgcgggaatggcaagcagtgaggagtcgaagcggtcgagcatgccgccgtggccggggagcacatggccgctgtctttaataccagctgacgcttgaaaaggctttcgaccaggtcgccccaggtgccgaatacgcataccgtcaggcccagacccaaccagtggagaaggggcaaggtg

SEQ ID NO: 6 - DNA, 541 bp, bacteroides
gacaatgccgacaggatagccattggcataaacgggattcgacactgaaagacctacaacgttcttgaactttacgtaataggtattagtggattgaaaaacattgatacccttgaggaaattgagtcctataaacagcagcacaagggctactacagcggtaagggcaattttaatttctttcttcattgtatggtgaatgaggttgtacttaaaggattgggatgcccatgatatggggggagtggtggcgcagaagcgtagggcttacttgcgttcggctgcacgggcttgcttaatggttactttctgaccattgagaaaggctaccatataggcatcggggagtttcttcaacactccgggcatggcgcgatgtgcttcggcatagttcgtgaacgaaccgtgatagtatttatagaggttgccttctttcactcgctttacggtgagtcccttgaactgggggtcgcggggcgaaatttcgcgagttccggctgccagttgaatgcagtaggttacgacattgccgaccacggta

SEQ ID NO: 7 - DNA, 376 bp, bacteroides
cggtgagctgattggtacaggatttgaccgaaactatgaaggtcttaccggagatatcgcctacaatccgcagctacagcgtgctgcttgtgtagacatccgctacacattgtttatcattgataaattcgcaggtgcacgccatctcatcgatgaaatgacaatcatccgataaggttgattttaccataaaaaaaagacatcctacctgattacccgacataagtctggggtttatcaaaagtaggatgtctttttttatactatcctgtaaaaaagaaaaagattathgcttaatgcttgaatcgtatgacgcacacgttctgcagtagttacgggatatacatattcatcttggtctgaactgcccagggta

SEQ ID NO: 8 - DNA, 709 bp, bacteroides
tatctatccgccataaagatgcatttggcgcaggaggaagtaccagcggtggtgtagatatacgccctttcccaaacaactgggagatgaaaaagcaacttggtgaggaaaaggagaatcaaacccgctgggacaatcatacagtaatgactcaacttgacaaaatcaatccgggtgatttggctttaatcatagattgcggttcagaggatttctttctcgaagtaaacgagcagctgcataaggttctgagtgacaaaggtattccgcatgacttcatcatccgcccgggagtacacgacgggaaatactggaataactcgattgattaccaatggatgtttttcaagaagttcttcgatggataccgcaataatcaagacatacgcccgtaggtaaaggcatctctccccaaccaaggtgccgataaagggatatacagcttaaatacataggtaaacttgccaagaagaaggtattgattatttcaaggctttttgtaaatttgcaattatatactcagaaaaagaacgataatatggaaataaccagtgctgaatttgtagtaagtaactcgcgtgccgacatgtgcccggacacccaccttcccgaacatgcctttatcggacgctcgaacgtaggagaatccagcttaatcaatatgctgaccaatcactccaatttggccatgacctcctcaaccccggta

SEQ ID NO: 9 - DNA, 285 bp, bacteroides
cacggtaaaaaacggactgactcccttcgcttcactgatttcagcaagtggaatatcatgtgtaataatagaagggaagaaagcttcgcctaccccgtaagaaagaaaacctgaaaacagcgttgctccataagccaacaaagcggttactgccaacatctttccggctcctttacctatatcggcaatggctggggtaactaatccgactataatcagcggaatgataaaattaaggaactgactgaacaacccgttgaaggtgacaaaaaggcgtacccggtg

SEQ ID NO: 10 - DNA, 576 bp, bacteroides
ttgagcttcttccagttagacgaccctgtgctttgtcagattcgtgatgaaatcttgaatttggatgtgaataatctgactcctctggaggcactgaataagttgaatgatattaagcggattgtaaggggaaagtagacttataaatcataaacttaaaaacgaaagccctatgaaaacaatgaagttaggacttcttcctcccattcttattacgatagttttaggtatttttgccgggatggtttcaccagctttcccggtacgcctttttgtcaccttcaacgggttgttcagtcagttccttaattttatcattccgctgattatagtcggattagttaccccagccattgccgatataggtaaaggagccggaaagatgttggcagtaaccgctttgttggcttatggagcaacgctgttttcaggttttctttcttacggggtaggcgaagctttcttcccttctattattacacatgatattccacttgctgaaatcagtgaagcgaagggagtcagtccgttttttaccgtgactattcctgagcccctgaatgtgataacatggca

SEQ ID NO: 11 - DNA, 392 bp, bacteroides
gaacgacccttcgcgtccgtgtcataaaacctacaccatcgacatggaccgtcactcgtacgatggccagcaatggacttacctgaacctgccgatggaggaaatcaagcagatggccattgcctcgattaaggactcaacgatgatgtactactcatgcgacgtgggcaaattcctgaacggcaaaacgggtattctttcgttgcagaactacgactacgagtcgcttttcggcaccaacttccccatgacgaaggctgaacgcatcagcacctttgcatcggcgtcgagccatgccatgacgctgatggccgtagacctcgacgccaacggcaaaccgacgaagtggatggttgaaaactcttggggtgctacttcgggccacaacggta

(19) United States
(12) Patent Application Publication
(10) Pub. No.: US 2018/0030518 A1
(43) Pub. Date: Feb. 1, 2018

(54) HOST-ASSOCIATED DNA SEQUENCES, PRIMERS, AND PROBES FOR PCR-BASED IDENTIFICATION OF DOG FECAL POLLUTION SOURCES

(71) Applicant: U.S. Environmental Protection Agency, Washington, DC (US)

(72) Inventors: Orin C. SHANKS, Cincinnati, OH (US); Hyatt C. GREEN, Cincinnati, OH (US)

(73) Assignee: U.S. Environmental Protection Agency, Washington, DC (US)

(21) Appl. No.: 15/783,595

(22) Filed: Oct. 13, 2017

Related U.S. Application Data
(63) Continuation-in-part of application No. 14/231,296, filed on Mar. 31, 2014, now Pat. No. 9,816,141.

(51) Int. Cl. C12Q 1/68

(52) U.S. Cl. CPC C12Q 1/689; C12Q 2600/158

(57) ABSTRACT

A method for detecting dog-fecal contamination in a sample, comprising assaying the sample using a nucleotide sequence based genetic assay which comprises contacting the sample with at least one nucleic acid molecule having the nucleic acid sequence shown in SEQ ID NO: 1, 2, 3, 4, 5, 6, 7, 8, 9, 10, 0111, the nucleic acid sequence being capable of binding to a nucleic acid sequence in the sample, and detecting binding of the nucleic acid molecule to the nucleic acid sequence in the sample, wherein a presence of binding is indicative of the presence of dog-fecal contamination in the sample; the nucleic acid molecules; and a kit comprising at least two of the above-described nucleic acid molecules.

### CROSS-REFERENCE TO RELATED APPLICATIONS

This application is a continuation-in-part of U.S. patent application Ser. No. 14/231,296, filed Mar. 31, 2014 in the U.S. Patent and Trademark Office. All disclosures are incorporated herein by reference.

### BACKGROUND OF THE INVENTION

Current regulatory methods used to assess microbial water quality rely on measuring the levels of culturable fecal indicator bacteria such as Enterococci and other fecal coliforms. However, the plate culture approach cannot discriminate among different specific bacterial strains or animal sources of fecal contamination.

Many studies have reported on the development of MST methods designed to target chemical indicators such as pharmaceuticals or the cultivation fecal bacteria isolated from particular animal sources. However, strategies that target genetic material isolated from fecal bacteria are the more widely reported. For example, ribotyping and rep-PCR DNA fingerprint techniques targeting E. coli isolates have been applied to discriminate among different animal fecal sources. However, the successful application of these genotypic methods depends on the development of large fingerprint databases of indicator bacterial isolates, primarily E. coli. Moreover, the use of E. coli for fecal source identification has been recently criticized in light of the abundance of secondary habitat populations that are capable of adapting to conditions outside of the animal gut and, as a result, contribute to the levels of fecal indicator bacteria in water.

Recently, Field and coworkers used library-independent methods based on ribosomal 16S rRNA gene sequences of Bacteroides-like bacteria to discriminate between human and ruminant feces. These Bacteroides markers have been used to identify non-point sources of fecal pollution in coastal and inland waters. However, these approaches often cannot not discriminate between fecal pollution sources at a species levels. For example, these methods can only discriminate between ruminant and non-ruminant animal sources due to the lack of genetic variation between Bacteroides 16S rRNA gene sequences originating from closely related animal species such as cows, sheep, deer, and other ruminants. Alternative MST methods capable of species level discrimination may prove more useful for fecal pollution characterization in recreational waters.

Functional genes involved in host-microbial interactions may represent a good pool of targets for host-specific assays. Some of these functional genes are hypothesized to be microbial surface proteins, while others may be associated with cellular processes and metabolism. However, a limited number of studies have used genes involved in host-microbial interactions as potential fecal community markers. This is probably due to the small number of microbial genes known to be involved in host-microbial interactions and the limited sequence information for these genes.

There is a demand for accurate microbial source tracking (MST) methods, because of language in the U.S. Clean Water Act regarding total maximum daily loads (TMDLs) and protection of supplies of drinking water. Current PCR-based MST approaches focus on various specific known DNA sequences, mostly targeting 16S rRNA (rDNA) genes, once thought to be source specific. However, validation studies are constantly uncovering exceptions and limitations with existing MST technologies. A significant part of the problem with existing 16S rRNA gene targeting MST methods stemmed from the inability to target microorganism DNA sequences encoding for proteins directly involved in host-microbe interactions, which are expected to contain high levels of genetic variation related to survival within different animal hosts.

Many specific approaches have previously attempted to determine sources of fecal contamination in the environment. One of the most widely used techniques is a PCR-based method that identifies ruminant fecal pollution by targeting bacterial 16S rRNA gene sequences from Bacteroides (Bernard and Field, AEM 66:4571-4574, 2000). The present inventors have conducted ongoing validation studies of these methods, and have discovered that previously described proposed ruminant specific markers can amplify rRNA gene sequences from non-ruminant fecal samples collected from geographic regions outside the original watersheds sampled. By definition, these previously described PCR target regions identify cow, deer, elk, goat, sheep, and other ruminants and pseudo-ruminants. This approach is therefore less useful in watersheds impacted by more than one ruminant animal source.

While advances in DNA sequencing and computational biology allow scientists to compare entire microbial genomes and discern microorganism-specific genetic information, sequencing of multiple closely related bacterial genomes so far remains prohibitively expensive and impractical for all but a very small number of laboratories. The entire genome content of more than 238 bacterial species have so far been defined through whole genome sequencing of representative type strains, and the number of genome sequences continues to increase. While significant differences in the genome content of different species are well-established, comparisons between genomes of closely related bacteria are equally important. These comparisons can provide species and strain-specific genetic information, define metabolic pathways and virulence factors, and provide insights into capacities for host-interactions, cell-to-cell signaling, stress response, and other essential microbial cellular functions.

Current DNA-based technologies potentially capable of identifying source, species, and strain-specific genetic markers include Suppressive Subtractive Hybridization (SSH) (Diatchenko et al., PNAS 93:6025-6030, 1996). This technique uses intentionally biased PCR amplification of nucleic acid pools to enrich for unique segments of restricted DNA relative to non-target DNA. SSH has been successfully applied in several pair-wise comparative genome studies (e.g., Nguyen et al., 2004, AEM 71 2564-2575), but only on one “metagenomic” or total microbial community DNA study (Galbraith et al., 2004; Environmental Microbiology: 928-937). SSH is a negative selection process that relies on unequal PCR amplification to amplify all dissimilar sequences from two nucleic acid pools. This is achieved by adding different self-complementary flanking regions to each of two fragment pools, and inhibition of amplification of only those duplexes that re-anneal relative to new heteroduplexes that form following denaturation and reassociation of the mixture.

One of the limitations of currently available MST methods arises from the inability of previously described techniques to target microorganism DNA sequences potentially encoding proteins directly involved in host-microbe interactions. These regions, unlike rRNA gene operons, are expected to retain high levels of genetic variation in microbes found in association with different animal hosts.

U.S. Pat. Nos. 7,572,584; 8,058,000, and 8,574,839 disclose positive DNA selection approaches designated Genome Fragment Enrichment (GFE) technique, and its efficient use in detecting fecal pollution from particular animal species and individual sources in environmental samples.

It is an object of the present invention to overcome the described deficiencies in the prior art.

### SUMMARY OF THE INVENTION

An embodiment of the disclosure comprises a method for detecting dog-fecal contamination in a sample, comprising assaying the sample using a nucleotide sequence based genetic assay which comprises contacting the sample with at least one nucleic acid molecule having the nucleic acid sequence shown in SEQ ID NO: 1, 2, 3, 4, 5, 6, 7, 8, 9, 10, or 11, the nucleic acid sequence being capable of binding to a nucleic acid sequence in the sample, and detecting binding of the nucleic acid molecule to the nucleic acid sequence in the sample, wherein a presence of binding is indicative of the presence of dog-fecal contamination in the sample.

An additional embodiment of the disclosure comprises the nucleic acid molecules having the nucleic acid sequences shown in SEQ ID NO: 1, 2, 3, 4, 5, 6, 7, 8, 9, 10, 11, or full complements thereof and capable of binding to a nucleic acid sequence in a sample containing fecal contamination.

A further embodiment of the disclosure relates to a kit comprising at least two of the above-described nucleic acid molecules.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 shows open reading frames identified from GFE.

### DETAILED DESCRIPTION OF THE EMBODIMENTS

Reference will now be made in detail to the present embodiments of the present invention, examples of which are illustrated in the accompanying drawings, wherein like reference numerals refer to the like elements throughout. The embodiments are described below in order to explain the present invention by referring to the figures.

There are approximately 78.2 million owned dogs in the United States. It is estimated that 39% of all households in the United States own at least one dog. Because these animals cohabitate with humans, it has been extremely difficult to develop technologies that can discriminate between human and dog fecal pollution. Dogs and their owners are known to frequent beaches, lakes, and parks situated near recreational water ways or storm sewage drains that discharge into local waters. Studies have shown that dog feces contains very high concentrations of enterocococi, a general fecal indicator bacteria recommend by the U.S. EPA for National Ambient Water Quality Criteria designed to keep the public safe from exposure to fecal pollution. Thus, it is impossible for a water manager to determine if high levels of general fecal indicators (i.e., enterococci) at a local beach where both human and dogs are present originates from humans, humans and dogs, or just dogs.

The host-associated technologies described here have several advantages over prior methods. First, our methods can differentiate between the select animal and other animal fecal pollution sources. Second, our methods target non-ribosomal genes predicted to be involved in host-associated metabolic activities. These genetic targets have never been used to develop host-associated fecal technologies for these animal groups. Third, our methods will work with populations from across the United States, where as it is currently unknown how effective prior art technologies are in this regard.

### EXAMPLE

Sample Collection. Fecal samples were collected over a wide geographic range. During transit from the field to the laboratory, samples were stored on ice and upon arrival, stored at −80° C. Sewage influent samples were also collected over a wide geographic range. Urban stormwater runoff samples were collected from a local rain garden over a 27 hour storm event. Water samples were retrieved within eight hours from the end of sampling and split for culture analysis of total coliforms and E. coli (MPN method, IDEXX Labs, Westbrook, Me.) and qPCR analysis. Automated sampler blanks were made by rinsing unused sample bottles with 50 ml distilled water prior to filtration. Filtrates from 50 ml of each stormwater sample were collected on 0.4 μM polycarbonate filters (Whatman, GE Healthcare Life Sciences, Piscataway, N.J.) and stored at −80° C. overnight prior to DNA extraction. A filter blank was performed in manner described above except that 50 ml of molecular grade water was used instead of stormwater.

Total DNA Extraction and Quantification. Both fecal and stormwater samples were extracted using the GeneRite DNA-EZ kit #DNA-EZ RWO2 [Shanks, O. C., et al., Competitive Metagenomic DNA Hybridization Identifies Host-Specific Microbial Genetic Markers in Cow Fecal Samples. Appl. Environ. Microbiol., 2006. 72(6): p. 4054-4060], North Brunswick, N.J.] according to the manufacturer's instructions with modifications. For fecal samples, about 0.5 g fecal sample was added to 1 ml GITC buffer and vortexed to make fecal slurries. Four hundred microliters elution buffer and 700 μl fecal slurry were added to the bead mill tube and agitated at 6 m/s for 40 sec. After centrifuging bead mill tubes for one minute at 14,000 g, 500 μl supernatant was combined with 1000 μl binding buffer and vortexed. This mixture was then added to the DNA binding column and washed twice with wash buffer. DNA was eluted with 100 μl warm elution buffer and stored at −20° C. until further analysis. DNA was extracted from water sample filtrates in the same manner except that 500 μl GeneRite lysis buffer and 12 ng Salmon sperm DNA was added to the tube with the filter instead of GITC and no elution buffer was added to the bead mill tube resulting in only 500 μl lysate being added to the bead tube before agitating. One or two extraction blanks were included with every extraction batch by adding 700 μl molecular grade water to the bead tube instead of fecal slurry or lysate for a total of 22 extraction blanks. Total DNA concentrations for each sample were estimated with PicoGreen (Life Technologies) following the manufacturers protocol on a SpectraMax® Paradigm® plate reader (Molecular Devices, Sunnyvale, Calif.).

Genome Fragment Enrichment and Enriched Sequence Annotation. GFE was performed as previously described [Shanks, O. C., et al., Identification of bacterial DNA markers for the detection of human fecal pollution in water. Appl Environ Microbiol, 2007. 73(8): p. 2416-22; Shanks, O. C., J. W. Santo Domingo, and J. E. Graham, Use of competitive DNA hybridization to identify differences in the genomes of bacteria. J Microbiol Methods, 2006. 66(2): p. 321-30; Shanks, O. C., et al., Competitive Metagenomic DNA Hybridization Identifies Host-Specific Microbial Genetic Markers in Cow Fecal Samples. Appl. Environ. Microbiol., 2006. 72(6): p. 4054-4060] using dog fecal total DNA as the target and swine fecal DNA as the blocker. Fragments were ligated into pCR4-TOPO plasmids and transformed into One Shot Top10 cells (Life Technologies, Carlsbad, Calif.). Amplicon Sanger sequencing was performed on an ABI PRISM 3730XL DNA Analyzer. Low quality sequence regions were removed by visual inspection. In some cases this resulted in discarding entire sequence fragments.

Predictions of possible gene function and taxonomic association were obtained through the RAMMCAP [Li, W., Analysis and comparison of very large metagenomes with fast clustering and functional annotation. BMC Bioinformatics, 2009. 10: p. 359] via CAMERA [Sun, S., et al., Community cyberinfrastructure for Advanced Microbial Ecology Research and Analysis: the CAMERA resource. Nucleic Acids Res, 2011. 39(Database issue): p. D546-51] and MG-RAST [Meyer, F., et al., The metagenomics RAST server —a public resource for the automatic phylogenetic and functional analysis of metagenomes. BMC Bioinformatics, 2008. 9: p. 386] pipelines, respectively. Identification of unique sequences was performed with CD-HIT using a 90% identity threshold [Li, W. and A. Godzik, Cd-hit: a fast program for clustering and comparing large sets of protein or nucleotide sequences. Bioinformatics, 2006. 22(13): p. 1658-9]. Identification of open reading frames (ORFs) was performed with Metagene [Noguchi, H., J. Park, and T. Takagi, MetaGene: prokaryotic gene finding from environmental genome shotgun sequences. Nucleic Acids Res, 2006. 34(19): p. 5623-30]. Marker selection by function was based on annotations using the Clusters of Orthologous Groups (COG) database [Kristensen, D. M., et al., A low-polynomial algorithm for assembling clusters of orthologous groups from intergenomic symmetric best matches. Bioinformatics, 2010. 26(12): p. 1481-7; Tatusov, R. L., E. V. Koonin, and D. J. Lipman, A genomic perspective on protein families. Science, 1997. 278(5338): p. 631-7]. Since database content has changed significantly since the time of initial fragment isolation and sequencing, more recent and perhaps more accurate annotations of selected marker fragments using BLASTx and the NCBI Protein Reference Sequence database were conducted.

Selection of Candidate Sequences for PCR-Based Assay Development. Sequences without statistically significant homology to sequences stored in sequence databases or the order Bacteroidales (e-value ≧0.001) were not selected for marker development. Thirty-eight sequences distributed across all functional categories were selected for marker development including sequences with hypothetical functions only. Forty-six sequences attributed to Bacteroidales bacteria were randomly selected for primer design. In some cases, a single sequence was used to design more than one primer set. In all, 84 sequences were used to design 92 primer sets for further testing.

Oligonucleotide Design and Preparation. Unique regions in putative marker sequences were selected for primer design by comparison with existing sequence information. Primer-BLAST was used to perform in silico tests to predict target specificity and melting temperature (60±2° C.). For each TagMan® qPCR assay, two probes were designed to target short sequence regions with high amino acid conservation. The probe which resulted in the highest sigmoidal efficiency across a canine fecal DNA dilution series was selected for further testing.

Standard Curve Generation. To generate standard curves, decimal dilutions of a custom plasmid (5×10⁻⁴ to 5 copies/μl) that contained target sequence regions of all canine-associated assays (IDT, San Jose, Calif.) were used as template for each assay. Similar plasmid constructs were used to generate standard curves for HF183/BacR287 and GenBac3. Data from six separately run standard curves (duplicate reactions at each concentration) were compiled by pooling data from each run for each assay using simple linear regression.

PCR and qPCR. Takara Ex Taq® Hot Start Version PCR reagents (Clontech Labs, Mountain View, Calif., USA), 200 nM each primer, 4.0 ng BSA, 2 μl template DNA, and molecular grade water were used for all endpoint PCR reactions. PCR products were visualized using 2% agarose gels with lithium borate buffer and 1× GelStar™ (Lonza, Allendale, N.J., USA). Unless otherwise stated, all endpoint PCR reactions were run for 35 cycles on a Tetrad 2 thermal cycler (Bio-Rad Laboratories, Hercules, Calif.). Twenty five microliter qPCR reactions consisted of 1× TaqMan® Environmental Master Mix (Life Technologies), 5.0 ng BSA, 1400 nM each primer, 100 nM probe (TaqMan® reactions only), 0.1× SYBR® Green 1 Dye (SYBR® Green reactions only; Life Technologies), 2 μl template DNA, and molecular grade water. All qPCR reactions were run for 40 cycles on a StepOne Plus instrument (Life Technologies). Four reaction replicates were run for each DNA sample. Reaction fluorescence from cycles eight to thirteen was used to establish baseline fluorescence and a fluorescence threshold of 0.03 was used for all canine qPCR assays. A minimum of two no template control reactions were included with each qPCR instrument run. TaqMan® qPCR assays HF183/BacR287 (in press), GenBac3, and Sketa22 were used as previously described.

Assay Selection. In order to determine which assays have diagnostic fecal source identification potential, the 92 primer sets were initially tested with endpoint PCR on two fecal DNA composites. One, a canine fecal DNA composite (0.05 ng total DNA/μl) consisting of equal DNA masses from 20 canine individuals (10 each from Florida and Wyoming populations) were used to determine the presence or absence of markers within the target host group. Two, a DNA composite consisting of human, cattle, and goose fecal DNA (0.625 ng total DNA/μl, equal DNA mass per source species) was used to determine the presence of markers in likely sources of contaminants other than dogs. For the initial assay screening phase only, the number of endpoint PCR amplification cycles was decreased from 35 to 30 when using canine fecal DNA as template to select for markers with high abundance in dogs. Assays were discarded from further analysis if any of the following conditions were met: 1) an amplification product of the expected size was absent when using canine fecal DNA as template, 2) amplification products of any size were produced when the non-canine fecal DNA composite was used as template, or 3) amplification byproducts, such as primer dimerization molecules or other spurious PCR products noticeably different in size from the expected PCR product, were produced when using either fecal DNA composite as template. Primer sets that met these criteria were used for more rigorous endpoint PCR testing against fecal DNA extracts listed in Table 1.

For this second phase of testing, non-canine fecal DNA composites consisting of 0.5 ng DNA μl⁻¹ individual⁻¹ were used to characterize marker distributions outside canine populations. Individual canine DNA extracts (0.5 ng/μl) were used to investigate the marker distributions within canine populations. Assays that had the highest estimates of specificity and maintained high levels of sensitivity after this second phase of screening were used as the basis for three qPCR assays.

SYBR® qPCR assays were created by incorporating each forward and reverse primer into a SYBR® Green qPCR chemistry platform without any modifications. The same was done for TagMan® qPCR assays, but with the addition of an internal probe and exclusion of SYBR® Green Dye. qPCR assays were then optimized for maximum specificity and efficiency (sigmoidal) by testing a range of primer (200-4000 nM) and probe (40-180 nM) reaction concentrations. At least two probes were tested for each TagMan® qPCR assay and the probe that resulted in the highest sigmoidal efficiency while maintaining specificity was chosen for further study. Amplification Interference and DNA Recovery Controls. Kinetic outlier detection (KOD) was used to check for amplification interference in all samples tested [Green, H. C. and K. G. Field, Sensitive detection of sample interference in environmental qPCR. Water Res, 2012. 46(10): p. 3251-60; Tichopad, A., et al., Quality control for quantitative PCR based on amplification compatibility test. Methods, 2010. 50(4): p. 308-12]. Briefly, genomic (Sketa22 only, 1-10⁻⁵ ng/rxn) or plasmid (all other assays, 10⁵-10¹ copies/rxn) DNA dilutions were used to generate a KOD reference data set. Then, reference and experimental fluorescence data were fit to a seven-parameter sigmoidal model and the linear relationship between fractional cycle values at first and second derivative maxima were used to estimate normal and outlier amplification profiles. Reactions with τₙₒᵣₘ values less than the 99.9% quantile using χ² distribution with one degree of freedom (-15.13) were considered significantly inhibited, but samples were not excluded from analysis unless all replicate reactions for a particular sample exceeded this threshold. All samples that amplified fit sigmoidal models with R²>0.999. The two samples containing levels of inhibitors were considered positive but non-quantifiable, and were excluded only from quantitative analysis. For stormwater samples, both DNA recovery and amplification interference was estimated with the Sketa22 assay similar to methods described previously [Green, supra]. Briefly, 12 μg Salmon sperm DNA was added to the sample before DNA extraction instead of E. coli AF504 GFP cells. Sketa22 amplification profiles were first checked for amplification inhibition using the methods described above. Amplification profiles that passed amplification criteria were then used to estimate total DNA recovery as a percentage of the total spiked salmon sperm DNA.

Data Analysis and Performance Parameter Definition. In R, package qpcR [Ritz, C. and A. Spiess, qpcR: an R package for sigmoidal model selection in quantitative real-time polymerase chain reaction analysis. Bioinformatics, 2008. 24: p. 1549-1551] was used for sigmoidal model fitting (functions modlist and perbatch) and package epitools [Liu, W. and D. A. Saint, Validation of a quantitative method for real time PCR kinetics. Biochemical and Biophysical Research Communications, 2002. 294(2): p. 347-353] was used to estimate binomial confidence intervals (function binom.exact). Marker prevalence was defined as the proportion of individuals that contained a marker. Specificity was defined as the proportion of non-canine samples testing negative for a canine marker. Sensitivity was defined as the proportion of canine samples testing positive for a canine marker. Sigmoidal efficiency, defined as the cycle specific efficiency (Eₙ) at the second derivative of the fitted amplification curve where Eₙ=Fₙ/(Fₙ₋₁) and Fₙ and Fₙ₋₁ are the cycle specific fluorescence values at cycles n and n−1 [Liu, W. and D. A. Saint, Validation of a quantitative method for real time PCR kinetics. Biochemical and Biophysical Research Communications, 2002. 294(2): p. 347-353], was used to estimate amplification efficiency during assay optimization. Amplification efficiency estimates from calibration curves were obtained using the equation E=10^{(1/-slope)}/2. Limit of quantification (LOQ) was defined as the lowest concentration of plasmid standard whose resulting mean Cq value fell within a linearity (R²>0.97). Cq_{LOQ} was defined as the mean Cq value at the LOQ. For TaqMan® reactions, the assay LOQ (10 plasmid copies/reaction, lowest concentration tested) was also considered the assay limit of detection (LOD). However, since SYBR® Green melt curve analysis could reliably distinguish smaller amounts of amplified target template from non-specific amplification products, any set of replicate reactions whose melt curves revealed the intended PCR product, regardless of their Cq values or the presence of side-product melt temperature (Tₘ) peaks, was considered to be in the detectable range. Thus, SYBR® reactions were interpreted as non-detections when a target Tₘ peak was absent in any of the replicate reactions. SYBR® reactions with the single intended Tₘ peak and Cq values within the LOQ were interpreted as quantifiable. Experimental Tₘ peaks within four standard deviations of the anticipated assay specific Tₘ peak (estimated using reference plasmids) were classified as target Tₘ peaks. Loads were estimated by first assuming that samples whose marker concentrations were below LOQ contained one marker copy. Loads of each sample were then averaged to obtain mean loads.

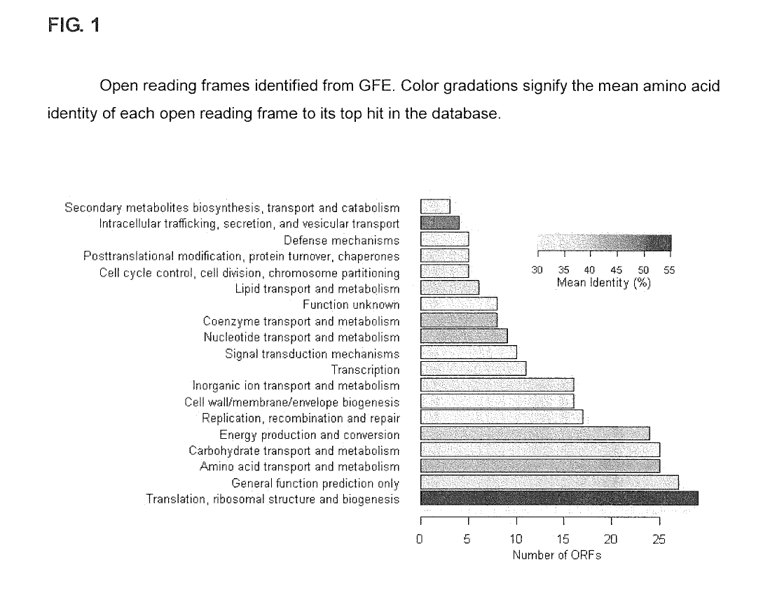

Annotation of Putative Canine-Associated DNA Sequences: Enriched sequence fragments were highly diverse in sequence composition and annotated function. Overall, sequences had very low similarity to reference database sequences, which made functional annotations difficult in some cases. Only 59.8%, 33.3%, and 41.4% of the 747 putative open reading frames (ORFs) had significant matches when searched against TIGRFAM, PFAM, and COG databases as of Nov. 11, 2011 (e-value ≦0.001), respectively. ORFs predicted to encode for genes involved in translation, ribosomal structure, and biogenesis were the most abundant (FIG. 1). Color gradations signify the mean amino acid identities of each open reading frame to its top hit in the database within each functional group.

Development of Canine-Associated Endpoint PCR Assays: Eighty-four sequences were selected for the development of 92 endpoint PCR assays based on functional and taxonomic annotations. Each endpoint PCR assay was tested using canine and mixed source fecal DNA composites. Twelve endpoint PCR assays (Table 1) were deemed eligible for sensitivity and specificity testing because they amplified a single expected PCR product from a canine fecal DNA composite and did not amplify PCR products from a non-canine fecal DNA preparation.

TABLE 1
Fragments annotations for which endpoint PCR assays were developed were
used to query the NCBI Protein Reference Sequence Database using BLASTx.

| | | | | | % | % | |
|---|---|---|---|---|---|---|---|
| | | | | | Query | Sequence | |
| Assay | Length | Taxonomy | Function | Accession | Coverage | ID (AA) | E-value |
| DG 2 | 676 | [symbol] | long-chain fatty | WP_{—}007559956.1 | 98 | 89 | 4.00E−12 |
| | | | acid-- | | | | |
| DG 3^{α} | | | CoA ligase | | | | |
| DG 5 | 355 | Caldanaerobacter | hypothetical | WP_009610708.1 | 59 | 92 | 1.00E−22 |
| | | subterraneus | protein | | | | |
| DG 29 | 550 | Lachnospiraceae | anti-sigma F | WP_003022855.1 | 56 | 77 | 3.00E−49 |
| | | | factor | | | | |
| | | | antagonist | | | | |
| DG 37 | 416 | [symbol] | cell division | WP_{—}003021240.1 | 71 | 89 | 4.00E−43 |
| | | | protein FtsY | | | | |
| DG 39 | 388 | Alloprevotella | phosphatidate | WP_006254289.1 | 45 | 90 | 5.00E−29 |
| | | tannerae | cytidylyltransferase | | | | |
| DG 46 | 541 | Prevotella sp. | hypothetical | WP_009436627.1 | 33 | 74 | 1.00E−16 |
| | | oral taxon | protein | | | | |
| | | 473 | | | | | |
| DG 68 | 377 | Bacteroides | hypothetical | WP_007837027.1 | 44 | 95 | 2.00E−29 |
| | | dorei | protein | | | | |
| DG 72 | 709 | [symbol] | hypothetical | WP_{—}007571683.1 | 52 | 77 | 8.00E−63 |
| | | | protein | | | | |
| | | | (esterase) | | | | |
| DG 74 | 285 | Bacteroides | sodium:proton | WP_007562866.1 | 97 | 86 | 2.00E−45 |
| DG 75 | 576 | plebeius | antiporter | | 69 | 83 | 1.00E−64 |
| DG 80 | 392 | Prevotella sp. | putative | WP_009436828.1 | 99 | 77 | 4.00E−66 |
| | | oral taxon 473 | aminopeptidase E | | | | |

^{α}Endpoint, SYBR ® Green qPCR, and TaqMan ® qPCR assays designed for assays in bold. For all other assays only endpoint versions were designed.

Secondary screening using 199 reference fecal DNA extracts demonstrated that all 12 assays exhibited high levels of specificity ranging from 89% to 100% and sensitivity from 73% to 95% (Table 2).

TABLE 2
Estimates of specificity and sensitivity for 12 canine-associated endpoint-PCR assays.

| | Specificity | | Sensitivity | | | | | |
|---|---|---|---|---|---|---|---|---|
| Assay | Num. Pos, n = (133) | Estimate | LCI^{α} | UCI | Num. Pos, n = (66) | Estimate | LCI | UCI |
| DG2 | 2 | 0.98 | 0.95 | 1.00 | 52 | 0.79 | 0.67 | 0.88 |
| DG3 | 0 | 1.00 | 0.97 | 1.00 | 51 | 0.77 | 0.65 | 0.87 |
| DG5 | 5 | 0.96 | 0.91 | 0.99 | 51 | 0.77 | 0.65 | 0.87 |
| DG29 | 15 | 0.89 | 0.82 | 0.94 | 63 | 0.95 | 0.87 | 0.99 |
| DG37 | 0 | 1.00 | 0.97 | 1.00 | 56 | 0.85 | 0.74 | 0.92 |
| DG39 | 8 | 0.94 | 0.88 | 0.97 | 48 | 0.73 | 0.6 | 0.83 |
| DG46 | 3 | 0.98 | 0.94 | 1.00 | 48 | 0.73 | 0.6 | 0.83 |
| DG68 | 0 | 1.00 | 0.97 | 1.00 | 51 | 0.77 | 0.65 | 0.87 |
| DG72 | 0 | 1.00 | 0.97 | 1.00 | 50 | 0.76 | 0.64 | 0.85 |
| DG74 | 3 | 0.98 | 0.94 | 1.00 | 52 | 0.79 | 0.67 | 0.88 |
| DG75 | 1 | 0.99 | 0.96 | 1.00 | 52 | 0.79 | 0.67 | 0.88 |
| DG80 | 7 | 0.95 | 0.89 | 0.98 | 48 | 0.73 | 0.6 | 0.83 |

^{α}LCI and UCI are 95% lower and upper confidence intervals based on a binomial distribution, respectively.

Development of Canine-Associated qPCR Assays. The top performing endpoint PCR assays, DG3, DG37, and DG72 (Table 1), were modified to two qPCR platforms, SYBR® Green and TaqMan® qPCR. Amplification of plasmid DNA dilutions showed that these qPCR assays have high reproducibility and resolution (Table 3).

TABLE 3
Standard curve performance parameters.

| Assay | Chemistry | Intercept | Slope | Efficiency^{α} | R² | Cq_{LOQ} | Resolution^{β} | CV %^{δ} |
|---|---|---|---|---|---|---|---|---|
| DG3 | SYBR | 33.962 | −3.485 | 0.936 | 0.994 | 30.624 | 0.140 | 1.728 |
| | TaqMan | 37.904 | −3.633 | 0.885 | 0.996 | 34.378 | 0.120 | 1.238 |
| DG37 | SYBR | 34.564 | −3.574 | 0.905 | 0.992 | 30.951 | 0.158 | 1.891 |
| | TaqMan | 38.150 | −3.604 | 0.894 | 0.995 | 34.522 | 0.128 | 1.324 |
| DG72 | SYBR | 35.543 | −3.545 | 0.915 | 0.988 | 32.062 | 0.192 | 2.210 |
| | TaqMan | 39.558 | −3.512 | 0.926 | 0.991 | 36.014 | 0.172 | 1.784 |

^{α}Efficiency = 10^{(-1/slope)} − 1
^{β}Resolution as estimated in Ruijter et al. 2013.
^{δ}Mean CV % indicates percent coefficent of variation across all five dilutions for each assay.

The estimated marker log₁₀ copies per ng of total DNA from the analysis of 244 fecal DNA extracts demonstrate a high abundance in dogs and extremely low in all other animal sources (Table 4).

TABLE 4
Estimated concentrations of canine-associated
markers in target and non-target fecal sources

Marker concentrations per ng DNA

| | Mean | StdDev |
|---|---|---|
| DG3SYBR | 3.08 | 0.64 |
| DG3TaqMan | 3.14 | 0.73 |
| DG37SYBR | 2.09 | 0.53 |
| DG37TaqMan | 2.24 | 0.53 |
| DG72SYBR | 3.08 | 0.69 |
| DG72TaqMan | 3.47 | 0.64 |

Detection of Canine-Associated qPCR Genetic Markers in Stormwater Samples:

The prevalence of canine-associated markers among urban stormwater samples was low (Table 5). Quantifiable canine marker concentrations were between 15 and 115 copies per reaction. All three canine markers were found in the sample with the highest counts of E. coli and no markers were found in samples with fewer than 144 E. coli MPN/100 ml. Concentrations of molecular markers did not correlate with culturable bacteria counts (p>0.4). The detection of canine-associated markers in 5 out of 18 urban stormwater samples suggests that they persist long enough under environmental conditions to be useful for water quality applications, and that they are unlikely to be ubiquitous or native in the environment.

TABLE 5
Quantification and detection of canine-associated genetic markers in stormwater samples

| | Total | | DG3 | | | | DG72 | | |
|---|---|---|---|---|---|---|---|---|---|
| Sample | Coliforms^{α} | E. coli | SYBR ^{β} | TaqMan | DG37 | SYBR | TaqMan | SYBR | TaqMan |
| 1 | 3.322 | 2.041 | − | − | | − | − | − | − |
| 2 | 3.489 | 2.033 | − | − | | − | − | − | − |
| 3 | 3.220 | 1.933 | − | − | | − | − | − | − |
| 4 | 3.417 | 2.314 | 1.60 | 1.17 | | − | − | 1.52 | 2.06 |
| 5 | 3.394 | 2.253 | − | − | | − | − | − | − |
| 6 | 3.441 | 1.890 | − | − | | − | − | − | − |
| 7 | 2.199 | 2.161 | + | − | | − | − | − | − |
| 8 | 3.352 | 2.746 | − | − | | − | − | − | − |
| 1 | 4.380 | 1.813 | − | − | | − | − | − | − |
| 2 | 4.380 | 2.464 | − | − | | − | − | − | − |
| 3 | 3.833 | 1.220 | − | − | | − | − | − | − |
| 4 | 3.614 | 0.716 | − | − | | − | − | − | − |
| 5 | 4.513 | 2.664 | − | − | | − | − | − | − |
| 6 | 3.629 | 3.029 | − | − | | − | − | + | − |
| 7 | 4.107 | 3.312 | − | − | | − | − | − | − |
| 8 | 4.489 | 2.730 | − | − | | − | − | − | − |
| 9 | 4.352 | 3.664 | 1.34 | − | | + | − | 1.54 | 1.81 |
| 10 | 4.352 | 3.489 | + | − | | − | − | − | − |

All quantities are logic₁₀ transformed.
“+” indicates detection below the LOQ.
“−” denotes below LOD.
^{α}Total coliforms and E. coli MPN/100 ml were estimated using IDEXX-MPN methods.
^{β} Units for marker concentration are copies/reaction

Although a few embodiments of the present invention have been shown and described, it would be appreciated by those skilled in the art that changes may be made in this embodiment without departing from the principles and spirit of the invention, the scope of which is defined in the claims and their equivalents.

SEQUENCE LISTING

>DogGFE_R2_1_F11_F_TQ_DG2_DG3
CCCTTCGTAATAGTTCCGCCACGAAGCAAGATTTCATTGTTTTCACCTAT
TTTAGCCTCTACTCCCGGAAGCAAACGTCCCACGGCACCGATGCGGAAAT
CATCTTTCCATTCACAAGACACCGTAGCCGTACTTTCTGTCAATCCATAG
CCGGCTACCATGTTAATACCTACCGAATGAACAAATTCAGCCACCTTTCC
AGGAATAGCCGCACCGGCAGTCGGGAAAAAGTTTCCGTTGTCAATTCCTA
TCGTCTTTTTCAGGAGGCTGTAAATCGTCCGTTCATAAAACTTATATTTC
ATATGCAACATCACGGGAGGAGTCTTACCATTCATCAGGTAATCAATGTT
ATGCGCCTTACCTACACGAAGGGCATCCAACATCAGACTCTTTTTCAGCC
CCGTTGTTTCGTTAATCTTTTCCAGTACGCCCGCGTAGACTTTTTCCCAG
AAACGGGGCACACTGCACATGGCTGTAGGGCGAACCTCCTTGATGGTTTT
CTGAATATCCTGCGGACGGAGGTTAATGCAGAGCAAACATCCACGGTCTA
AACAATAATATGACCATGCCCGCTCAAAAATGGTA
>DogGFE_R2_5_B05_M13R_TQ_DG5
GGCACGTAGAGAATACGAAGGTGAGCGAGAGAACTGTTGCCAAGGAACTC
GGCAAAATGGCCCCGTACGTTCGCAAGAAGGGGCGCTCGAGAGAGCCGCA
GTGAAAAGGCCCAAGCGACTGTTTAACTAAAACACAGCTCTATGCAAAGC
CGTAAGGCGAAGTATATGGGGTGACACCTGCCCGGTGCTGGAAGGTTAAG
GGGATCTGTTCGTCGCAAGACAAAGCAGTGGTCCGAAGCCCCAGTGAACG
GCGGCCGTAACTATAACGGTCCTAAGGTAGCGAAATTCCTTGTCGGGTAA
GTTCCGACCCGCACGAAAGGCGTAATGATTTGAGCGCTGTCTCGACAATC
CAGTG
>DogGFE_R2_2_E09_M13R_TQ_DG29
TGAAATAAGAACCAGGGAAAACCTTGAGGAAAAGGAGCTATCATAGCTGA
CGCATATCAGGAACACTGATACCGTTCACGCCCAGTCTGCGCTTCCTTTG
AGCCTGTAGTCTCAAAGGCGCTCGACGGATTCGCGTAAACGGTGTCAATG
TCCCGGATATGCTGTTGGCATGATAGCTCTTTGTGTATAGGTGTGCAGAT
GGAGAGTGTGTTTGAATAAATCGAAAAAGTGGAGGCGGATTATGGAAGAG
TGTATGACAAAGAGGGGAGCCAGGCTGATTGTGTATGTGCCTAAGGAGCT
GGATCATCATTTTGCAGGGGAGATGACAGAGATGGTGGATAAGGAGCTTA
AGAAGGGCTGTATTAAACAGCTTGTCTTTGATTTTTCTAAGACAACGTTT
ATGGACAGTTCTGGCATTGGGATGCTAATGGGGAGAAAGCGGCTGTTAAG
CTACAGCGGAGGAACCGTAAGTGCTATTCATGTTAATGACCGGATTTTGC
GGATTATGCAGCCTTCGGGCATCTGGAAACACATGGAAATCAGCCAGGAA
>DogGFE_R2_6_E05_M13F_TQ_DG37
TAGTCCTTTTCTCCCACGGTCATCTGCTCTTTCATACTGTCCATAAGAAG
CTGCTTACATTCCTGTGGATCCTTAATATGCTTTTCTGCCACCTGCTCCT
TTAAACGTTCAATAATAGAAGTGGTAGTATTAATTCCAATGTCGCCCATA
ACCAAGATTTCTTCGATTTCCTCATAGAAATCCTCGTCAATACTGGAATA
ACCGCTAAAAATAGAATCAATTCCTGATACAATGTTTTCTCTGGTTTTTG
AAAGACCCTCTACCAATCHTTAAAAAAACCTTTTTTCTCTTTTCCCTCTG
CCATAGTTCCTCCTATTTATCTAAATCATGCTCAATGAGTTTTACAGACA
CCAGGGTAGACACACCTTTTTCCTGCATGGTAATTCCATAGAGCCTGTCT
GCTGCTGTCATGGTT
>DogGFE_R2_3_B06_M13F_TQ_DG39
GTGCTGACCAAGGAGGTGAGAAGCAGCACTATCAAGGCAATTTTAAGAAT
CAGCTTGCGCCGATTGATTATGTCAGATGGATGAAGCATAGTGACTGTGT
AACTTAAATGGGTGTTCAGAATTCACACATTTGAACACTTGCTTGGGTAA
CTTGAACTTTAAACTCGATGCTTAAAACGCCTGAATGGTGTAAAGGTAAA
GAACGGCTGCGGGAATGGCAAGCAGTGAGGAGTCGAAGCGGTCGAGCATG
CCGCCGTGGCCGGGGAGCACATGGCCGCTGTCTTTAATACCCAGCTGACG
CTTGAAAAGGCTTTCGACCAGGTCGCCCCAGGTGCCGAATACGCATACCG
TCAGGCCCAGACCCAACCAGTGGAGAAGGGGCAAGGTG
>DogGFE_R2_3_F12_M13F_TQ_DG46
GACAATGCCGACAGGATAGCCATTGGCATAAACGGGATTCGACACTGAAA
GACCTACAACGTTCTTGAACTTTACGTAATAGGTATTAGTGGATTGAAAA
ACATTGATACCCTTGAGGAAATTGAGTCCTATAAACAGCAGCACAAGGGC
TACTACAGCGGTAAGGGCAATTTTAATTTCTTTCTTCATTGTATGGTGAA
TGAGGTTGTACTTAAAGGATTGGGATGCCCATGATATGGGGGGAGTGGTG
GCGCAGAAGCGTAGGGCTTACTTGCGTTCGGCTGCACGGGCTTGCTTAAT
GGTTACTTTCTGACCATTGAGAAAGGCTACCATATAGGCATCGGGGAGTT
TCTTCAACACTCCGGGCATGGCGCGATGTGCTTCGGCATAGTTCGTGAAC
GAACCGTGATAGTATTTATAGAGGTTGCCTTCTTTCACTCGCTTTACGGT
GAGTCCCTTGAACTGGGGGTCGCGGGGCGAAATTTCGCGAGTTCCGGCTG
CCAGTTGAATGCAGTAGGTTACGACATTGCCGACCACGGTA
>DogGFE_R3_8_C01_M13F_TQ_DG68
CGGTGAGCTGATTGGTACAGGATTTGACCGAAACTATGAAGGTCTTACCG
GAGATATCGCCTACAATCCGCAGCTACAGCGTGCTGCTTGTGTAGACATC
CGCTACACATTGTTTATCATTGATAAATTCGCAGGTGCACGCCATCTCAT
CGATGAAATGACAATCATCCGATAAGGTTGATTTTACCATAAAAAAAAGA
CATCCTACCTGATTACCCGACATAAGTCTGGGGTTTATCAAAAGTAGGAT
GTCTTTTTTTATACTATCCTGTAAAAAAGAAAAAGATTATTTGCTTAATG
CTTGAATCGTATGACGCACACGTTCTGCAGTAGTTACGGGATATACATAT
TCATCTTGGTCTGAACTGCCCAGGGTA
>DogGFE_R3_7_B02_M13F_TQ_DG72
TATCTATCCGCCATAAAGATGCATTTGGCGCAGGAGGAAGTACCAGCGGT
GGTGTAGATATACGCCCTTTCCCAAACAACTGGGAGATGAAAAAGCAACT
TGGTGAGGAAAAGGAGAATCAAACCCGCTGGGACAATCATACAGTAATGA
CTCAACTTGACAAAATCAATCCGGGTGATTTGGCTTTAATCATAGATTGC
GGTTCAGAGGATTTCTTTCTCGAAGTAAACGAGCAGCTGCATAAGGTTCT
GAGTGACAAAGGTATTCCGCATGACTTCATCATCCGCCCGGGAGTACACG
ACGGGAAATACTGGAATAACTCGATTGATTACCAATGGATGTTTTTCAAG
AAGTTCTTCGATGGATACCGCAATAATCAAGACATACGCCCGTAGGTAAA
GGCATCTCTCCCCAACCAAGGTGCCGATAAAGGGATATACAGCTTAAATA
CATAGGTAAACTTGCCAAGAAGAAGGTATTGATTATTTCAAGGCTTTTTG
TAAATTTGCAATTATATACTCAGAAAAAGAACGATAATATGGAAATAACC
AGTGCTGAATTTGTAGTAAGTAACTCGCGTGCCGACATGTGCCCGGACAC
CCACCTTCCCGAACATGCCTTTATCGGACGCTCGAACGTAGGAGAATCCA
GCTTAATCAATATGCTGACCAATCACTCCAATTTGGCCATGACCTCCTCA
ACCCCGGTA
>DogGFE_R2_4_G07_M13F_TQ_DG74
CACGGTAAAAAACGGACTGACTCCCTTCGCTTCACTGATTTCAGCAAGTG
GAATATCATGTGTAATAATAGAAGGGAAGAAAGCTTCGCCTACCCCGTAA
GAAAGAAAACCTGAAAACAGCGTTGCTCCATAAGCCAACAAAGCGGTTAC
TGCCAACATCTTTCCGGCTCCTTTACCTATATCGGCAATGGCTGGGGTAA
CTAATCCGACTATAATCAGCGGAATGATAAAATTAAGGAACTGACTGAAC
AACCCGTTGAAGGTGACAAAAAGGCGTACCCGGTG
>DogGFE_R2_5_D09_M13F_TQ_DG75
TTGAGCTTCTTCCAGTTAGACGACCCTGTGCTTTGTCAGATTCGTGATGA
AATCTTGAATTTGGATGTGAATAATCTGACTCCTCTGGAGGCACTGAATA
AGTTGAATGATATTAAGCGGATTGTAAGGGGAAAGTAGACTTATAAATCA
TAAACTTAAAAACGAAAGCCCTATGAAAACAATGAAGTTAGGACTTCTTC
CTCCCATTCTTATTACGATAGTTTTAGGTATTTTTGCCGGGATGGTTTCA
CCAGCTTTCCCGGTACGCCTTTTTGTCACCTTCAACGGGTTGTTCAGTCA
GTTCCTTAATTTTATCATTCCGCTGATTATAGTCGGATTAGTTACCCCAG
CCATTGCCGATATAGGTAAAGGAGCCGGAAAGATGTTGGCAGTAACCGCT
TTGTTGGCTTATGGAGCAACGCTGTTTTCAGGTTTTCTTTCTTACGGGGT
AGGCGAAGCTTTCTTCCCTTCTATTATTACACATGATATTCCACTTGCTG
AAATCAGTGAAGCGAAGGGAGTCAGTCCGTTTTTTACCGTGACTATTCCT
GAGCCCCTGAATGTGATAACATGGCA
>DogGFE_R3_7_H11_M13F_TQ_DG80
GAACGACCCTTCGCGTCCGTGTCATAAAACCTACACCATCGACATGGACC
GTCACTCGTACGATGGCCAGCAATGGACTTACCTGAACCTGCCGATGGAG
GAAATCAAGCAGATGGCCATTGCCTCGATTAAGGACTCAACGATGATGTA
CTACTCATGCGACGTGGGCAAATTCCTGAACGGCAAAACGGGTATTCTTT
CGTTGCAGAACTACGACTACGAGTCGCTTTTCGGCACCAACTTCCCCATG
ACGAAGGCTGAACGCATCAGCACCTTTGCATCGGCGTCGAGCCATGCCAT
GACGCTGATGGCCGTAGACCTCGACGCCAACGGCAAACCGACGAAGTGGA
TGGTTGAAAACTCTTGGGGTGCTACTTCGGGCCACAACGGTA

What is claimed is:

1. A nucleic acid molecule having at least one nucleic acid sequence shown in SEQ. ID NO. 1, 2, 3, 4, 5, 6, 7, 8, 9, 10, or 11 or any sequence which constitutes a full complement thereof.

2. A composition of matter comprising at least one molecule of claim 1 bound to a sequence in a sample containing dog fecal matter.

3. A composition comprising a sample of water to which has been added nucleic acid molecule having at least one the nucleic acid sequence shown in SEQ. ID NO. 1, 2, 3, 4, 5, 6, 7, 8, 9, 10, or 11 or a full compliments to said sequences.
